(19) United States
(12) Patent Application Publication
(10) Pub. No.: US 2022/0192849 A1
(43) Pub. Date: Jun. 23, 2022

(54) MAGNETIC CELLS FOR CONTROLLING THE SHAPE OF PIPE WITH FLUID FLOW, A METHOD FOR PRODUCING THEREOF, SYSTEM FOR CONTROLLING THE SHAPE OF A PIPE WITH FLUID FLOW AND ARTIFICIAL INTELLIGENCE PLANNING SYSTEM FOR CONTROLLING THE SHAPE OF PIPES WITH FLUID FLOW USING MAGNETIC CELLS

(71) Applicant: Industry-Academic Cooperation Foundation, Yonsei University, Seoul (KR)

(72) Inventors: Joon Sang LEE, Seoul (KR); Han Sung JUNG, Seoul (KR); Shinill KANG, Seoul (KR); Jung Sun KIM, Seoul (KR); Hyung Ju CHO, Seoul (KR); Yoon Jeong CHOI, Seoul (KR); Hwi Dong JUNG, Seoul (KR); Young Woo KIM, Seoul (KR); Susie RYU, Seoul (KR); Seungchul KO, Seoul (KR)

(21) Appl. No.: 17/457,624

(22) Filed: Dec. 3, 2021

(30) Foreign Application Priority Data
Dec. 4, 2020 (KR) 10-2020-0168665

(51) Int. Cl. A61F 2/82

(52) U.S. Cl. CPC A61F 2/82; A61F 2210/009

(57) ABSTRACT

Provided are a magnetic cell for controlling a shape of a pipe with fluid flow including a magnetic material in the cell, a method of producing the same, and a system for controlling a shape of a pipe with fluid flow and an artificial intelligence planning system for controlling a shape of a pipe with fluid flow using the same, and a repulsive force or attractive force between the magnetic cells is used to induce expansion or contraction of the pipe with fluid flow to control the shape of the pipe with fluid flow.

### CROSS REFERENCE TO RELATED APPLICATION

The present application claims priority to Korean Patent Application No. 10-2020-0168665 filed on Dec. 4, 2020. The entire contents of the above-listed application is hereby incorporated by reference for all purposes.

### TECHNICAL FIELD

The following disclosure relates to a magnetic cell for controlling a shape of a pipe with fluid flow, and more particularly, to a magnetic cell which expands or contracts a biological pipe with fluid flow such as a blood vessel or a respiratory tract, a method of producing the same, and a system using the same.

### BACKGROUND

In general, a stent is an artificial pipe structure intended for permanent insertion into an autologous or graft vascular or non-vascular system or the like, is mainly used for expansion of a stenosis site, and is manufactured for its use depending on the size, nature, and environment of various organs and lumens in which the stent is installed.

A stent to be applied to a coronary artery is manufactured using a stainless steel and a shape memory alloy as main materials and though it is inserted into a blood vessel in a nonoperative method, a small hole is made in the blood vessel to place a catheter inside the blood vessel, a contrast agent is injected to confirm the condition and position of a lesion in the blood vessel, a balloon catheter coupled with the stent for a coronary artery is placed in the lesion along a guidewire, and then the balloon is inflated to be pressed on a vascular lesion to secure the lumen of the blood vessel. During the process, an intimal injury may occur, and when a vascular muscle cell overgrows in the process of treating a damaged blood vessel, in-stent restenosis may occur.

A stent used in a urethra for improving dysuria and urethrophraxis due to prostatism is inserted for a short time as a minimally invasive procedure, but it has a problem of being out of the initial position in the procedure; for example, it slides when urinating or slides upward to be raised and placed in the bladder, has a discomfort problem such as a foreign body sensation and a feeling of having residual urine, and has a very narrow tube lumen to be easily clogged.

Obstructive sleep apnea is a disease in which when a muscle at the base of a tongue (soft palate) and a muscle of a tissue hanging in the center of the back of a uvula (pharyngeal wall) relax or sag, a tissue loosened during breathing vibrates with airflow to cause snoring and closes a respiratory tract to cause noisy breathing so that breathing stops. As a non-operative treatment, a positive pressure machine which is used for the disease by blowing air to increase the pressure in the respiratory tract to maintain proper pressure in the respiratory tract is only a temporary and auxiliary medical apparatus for symptom relief, and its constant wear may cause complications such as pneumonia, dry nasal passages, and muscle pain due to contamination.

Therefore, research for solving the problems described above and basically relieving stenosis of a blood vessel, a respiratory tract, and the like related to the symptoms of the above diseases is needed.

In addition, since the stent is individually manufactured depending on the size, nature, and environment of organs and lumens in which the stent is installed due to the different structure and elasticity of the pipe and the different nature of the fluid flowing in the pipe depending on the organ such as circulatory, respiratory, and urinary organs, the stents are currently incompatible. Thus, an approach toward the development of a technology to allow compatibility is needed by basically controlling the pipe shape.

### SUMMARY

### Technical Problem

An embodiment of the present disclosure is directed to freely control a shape of a biological pipe with fluid flow, without problems of a foreign body sensation and re-coalescence conventionally caused by the use of a stent, using a magnetic cell for controlling a shape of a pipe with fluid flow including a magnetic material in the cell.

Another embodiment of the present disclosure is directed to providing a magnetic cell for controlling a shape of a pipe with fluid flow without a side effect of immune rejection, by producing the magnetic cell using autologous cell which is easy to collect.

Another embodiment of the present disclosure is directed to providing a system for controlling a shape of a pipe with fluid flow which provides a pipe shape control effect using a magnetic cell and also further providing a treatment effect as a photothermal effect by near infrared irradiation.

Still another embodiment of the present disclosure is directed to providing an artificial intelligence planning system for controlling a shape of a pipe with fluid flow using a magnetic cell which allows injection optimized for each patent by determining an injection method, an injection amount, and an injection site of the magnetic cell using artificial intelligence.

### Solution to Problem

In one general aspect, a magnetic cell for controlling a shape of a pipe with fluid flow includes a magnetic material in the cell.

In an exemplary embodiment of the present disclosure, the cell may be an inner wall cell of a pipe with fluid flow.

In an exemplary embodiment of the present disclosure, the magnetic material may be a permanent magnet.

In an exemplary embodiment of the present disclosure, the magnetic material may be bound to a cell surface.

In an exemplary embodiment of the present disclosure, the magnetic material may be placed inside the cell.

In an exemplary embodiment of the present disclosure, the shape control may be expansion or contraction of the pipe with fluid flow using a repulsive force or attractive force between the magnetic cells.

In an exemplary embodiment of the present disclosure, the pipe with fluid flow may be a blood vessel, a urethra, a respiratory tract, or an esophagus.

In another general aspect, a method of producing a magnetic cell for controlling a shape of a pipe with fluid flow includes: preparing an inner wall cell of a pipe with fluid flow; coating a magnetic bead with an inner wall cell-specific antibody; fixing the cell to a mold so that a portion of the inner wall cell is exposed; treating a surface of the inner wall cell with the antibody-coated magnetic bead in a state in which a magnetic field is applied to the mold; and binding the inner wall cell and the antibody-coated magnetic bead.

In an exemplary embodiment of the present disclosure, preparing of the inner wall cell of a pipe with fluid flow may include dedifferentiating cells collected from a user into an induced pluripotent stem cell and differentiating the induced pluripotent stem cell into the inner wall cell of the pipe with fluid flow.

In an exemplary embodiment of the present disclosure, the magnetic bead may have a particle diameter of 50 to 1000 nm.

In another general aspect, a system for controlling a shape of a pipe with fluid flow includes the magnetic cell described above and a magnetic field application device.

In an exemplary embodiment of the present disclosure, the system for controlling a shape of a pipe with fluid flow may further include an optical coherence tomography (OCT) device to observe image information of a magnetic cell bound to a pipe with fluid flow in real time after applying a magnetic field by the magnetic field application device, thereby providing feedback information for controlling the shape of the pipe with fluid flow.

In an exemplary embodiment of the present disclosure, the system for controlling a shape of a pipe with fluid flow may further include a near-infrared irradiation device.

In another general aspect, an artificial intelligence planning system for controlling a shape of a pipe with fluid flow using a magnetic cell includes: a database unit which includes personal information, medical image information, and shape information of a pipe with fluid flow of existing patients with a disease in a pipe with fluid flow; an interface unit which receives input of biometric information, medical image information, and shape factors of the pipe with fluid flow of patients with a disease in a pipe with fluid flow and outputs predetermined results; a simulation unit which analyzes the information received from the database unit and the interface unit by an artificial intelligence technique to predict a patient's situation after injection of the magnetic cell; and a control planning unit which determines an injection method and an injection site of the magnetic cell depending on the results of the simulation unit and calculates an injection amount.

In an exemplary embodiment of the present disclosure, the injection method of the magnetic cell may be direct injection into an affected area.

In an exemplary embodiment of the present disclosure, the injection method of the magnetic cell may be inserting the magnetic cell into a soft foam and transplanting the foam into the pipe with fluid flow.

In an exemplary embodiment of the present disclosure, the disease in the pipe with fluid flow may be any one or more selected from the group consisting of carotid artery stenosis, cerebral aneurysm, dilated cardiomyopathy, abdominal aneurysm, iliac aneurysm, varicose veins, urethral stenosis, prostatic hyperplasia, coronary artery stenosis, asthma, obstructive sleep apnea, and chronic obstructive pulmonary disease.

In still another general aspect, a ferromagnetic cell cluster includes a plurality of paramagnetic cells, wherein the magnetic cells form a circular ferromagnetic cell cluster and the ferromagnetic cell cluster is attached to an elastic pipe inner wall to expand or contract a diameter of the elastic pipe by magnetic field application.

### Advantageous Effects

The present disclosure magnetically controls a shape of a pipe with fluid flow when a pipe with fluid flow in a circulatory system, a respiratory system, and urinary system is abnormally narrowed or expanded, and in particular, does not need several invasive procedures, by controlling the shape of the pipe with fluid flow using a permanent magnet, and uses an autologous cell to have no side effect such as immune rejection.

By injecting or transplanting the magnetic material included in the magnetic cell according to the present disclosure into a disease due to deformation of a pipe lumen shape, an attractive force or a repulsive force is generated in a desired direction to be used in the treatment of a disease in a pipe with fluid flow.

In addition, the present disclosure may be injected by easily adjusting a cell spreading rate and an injection amount of the magnetic cells depending on the severity of the disease of mild or severe disease, and may provide injection optimized to an individual patient such as an injection site and an injection amount of the magnetic cell according to artificial intelligence based on big data.

### BRIEF DESCRIPTION OF THE FIGURES

FIGS. 1A and 1B show a principle of controlling a shape of a pipe with fluid flow using a magnetic cell according to the present disclosure, in which FIG. 1A is a schematic diagram showing contraction of a pipe with fluid flow by a magnetic attractive force, and FIG. 1B is a schematic diagram showing expansion of a pipe with fluid flow by a magnetic repulsive force.

FIG. 2 is an in-vivo injection method of magnetic cells produced according to Example 1 of the present disclosure.

FIG. 3 is a schematic diagram showing a process of preparing an inner wall cell of a pipe with fluid flow required for producing the magnetic cell according to the present disclosure.

FIGS. 4A and 4B shows results of imparting ferromagnetism of the magnetic cell produced according to Example 1 of the present disclosure.

FIGS. 5A and 5B show an injection method of the magnetic cell of the present disclosure, in which FIG. 5A shows a liquid parenteral injection method, and FIG. 5B shows an injection method by soft foam transplantation.

FIG. 6 schematically shows an artificial intelligence planning system for controlling a shape of a pipe with fluid flow for injection optimized for each patient of the magnetic cell according to the present disclosure.

FIG. 7 shows a block diagram illustrating the artificial intelligence planning system for controlling a shape of a pipe with fluid flow using the magnetic cell.

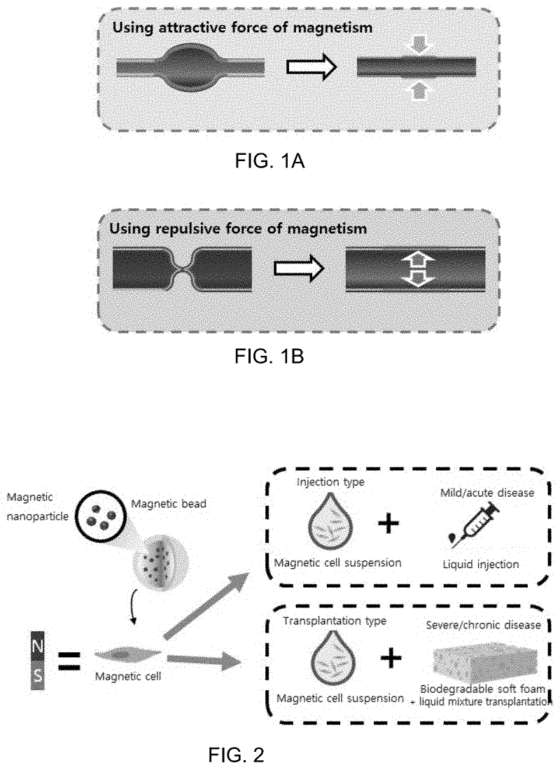

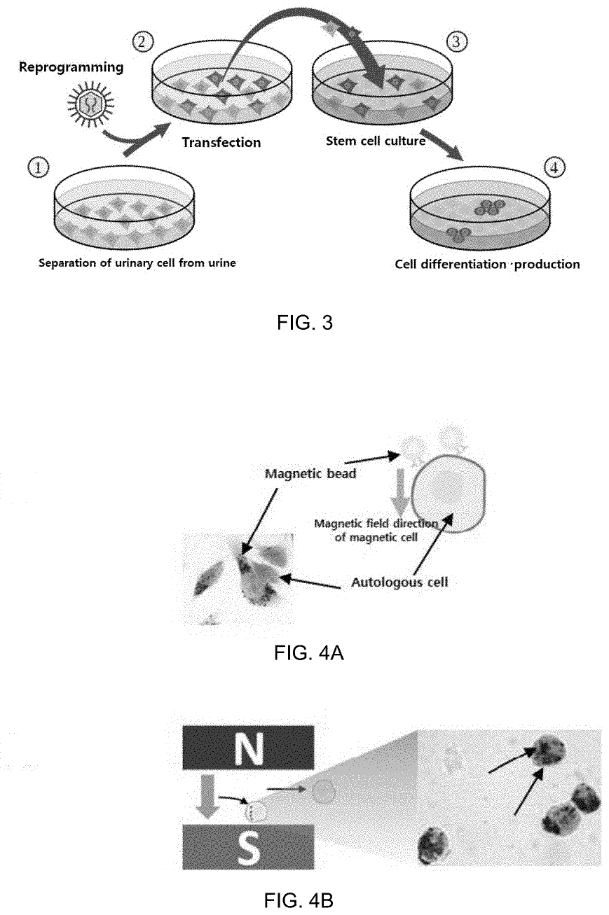

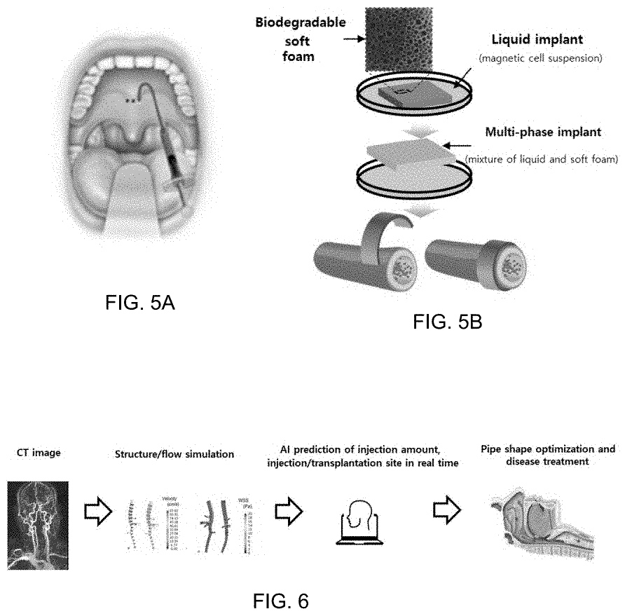

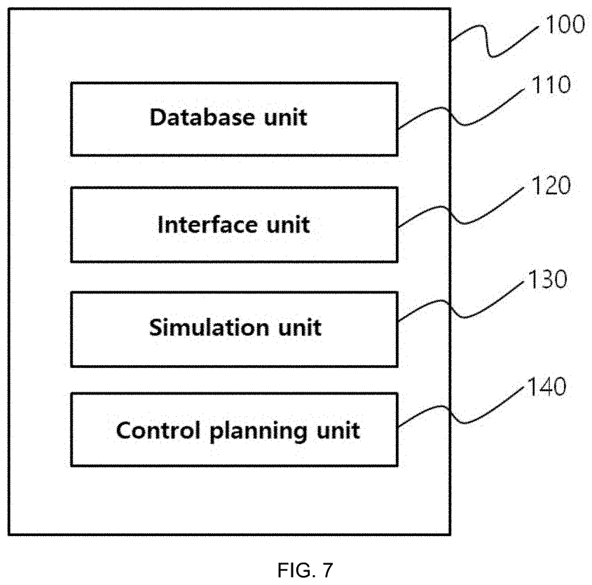

### DETAILED DESCRIPTION

Hereinafter, the magnetic cell for controlling a shape of a pipe with fluid flow according to the present disclosure will be described in detail. Herein, unless otherwise defined, all technical and scientific terms have the meaning commonly understood by those of ordinary skill in the art, and the terms used in the description of the present disclosure are only for effectively describing a certain example and are not intended to limit the present disclosure.

In addition, in the following description, description for well-known effects and configurations that may obscure the gist of the present disclosure unnecessarily will be omitted. Hereinafter, units used in the present specification without particular mention are based on weights, and as an example, a unit of % or ratio refers to a wt % or a weight ratio.

In addition, in describing constituent elements of the present disclosure, terms such as first, second, A, B, (a), and (b) may be used. These terms are used only to differentiate the constituent elements from other constituent elements, and the nature, sequence, order, or the like of the corresponding constituent elements is not limited by these terms.

In addition, the singular form used in the specification of the present disclosure may be intended to include a plural form, unless otherwise indicated in the context.

Hereinafter, the magnetic cell for controlling the shape of a pipe with fluid flow according to the present disclosure will be described in detail.

The term “pipe with fluid flow” used in the present specification is defined as a common name of a pipe composed of a soft tissue in which gas or liquid flows in the human body.

The present disclosure provides a magnetic cell for controlling a shape of a pipe with fluid flow including a magnetic material in the cell.

In an exemplary embodiment of the present disclosure, the cell may be an inner wall cell of the pipe with fluid flow. Herein, the pipe with fluid flow is a blood vessel, a urethra, a respiratory tract, or an esophagus, and the inner wall cell of the pipe with fluid flow may be, specifically, a pipe inner wall cell of a circulatory, urinary, or respiratory organ in the human body.

The present disclosure includes a magnetic material in the pipe inner wall cell, in which the magnetic material may be a paramagnetic, superparamagnetic, diamagnetic, or ferromagnetic material, but a ferromagnetic material which may become a permanent magnet by a magnetic field may also be used.

Specifically, for example, the magnetic material may be iron oxide, ferrite, or an alloy. Specifically, for example, the magnetic material may be maghemite (γ-Fe₂O₃), magnetite (Fe₃O₄), cobalt ferrite (CoFe₂O₄), manganese ferrite (MnFe₂O₄), an iron-platinum alloy (FePt alloy), an iron-cobalt alloy (FeCo alloy), a cobalt-nickel alloy (CoNi alloy), or a cobalt-platinum alloy (CoPt alloy), and the kind of magnetic material is not particularly limited thereto as long as it may become a permanent magnet.

The magnetic material may have an average diameter in a range of 1 to 1000 nm.

The magnetic material may be included in the cell, in the form of being bound to a cell surface or being placed inside the cell.

The shape control refers to expansion or contraction of the pipe with fluid flow using a repulsive force or attractive force between magnetic cells. Specifically, in an exemplary embodiment of the present disclosure, the magnetic material is a permanent magnet in which a ferromagnetic body is magnetized, and may be applied to a disease requiring expansion of the pipe lumen, by injecting magnetic cells into a pipe inner wall to appropriately arrange the cells so that all directions toward a pipe lumen are N-poles to cause the repulsive force of magnetism, and may be applied to diseases requiring contraction of the pipe lumen, by injecting magnetic cells in which the direction toward the pipe lumen are N-poles or S-poles and appropriately arranging the cells to cause the attractive force of magnetism.

The disease requiring contraction or expansion of the pipe with fluid flow may be, specifically, any one selected from the group consisting of carotid artery stenosis, cerebral aneurysm, dilated cardiomyopathy, abdominal aneurysm, iliac aneurysm, varicose veins, urethral stenosis, prostatic hyperplasia, coronary artery stenosis, asthma, obstructive sleep apnea, and chronic obstructive pulmonary disease, but is not necessarily limited thereto.

In addition, the present disclosure provides a method of producing a magnetic cell for controlling a shape of a pipe with fluid flow including: preparing an inner wall cell of a pipe with fluid flow; coating a magnetic bead with an inner wall cell-specific antibody; fixing the cell to a mold so that a portion of the inner wall cell is exposed; treating a surface of the inner wall cell with the antibody-coated magnetic bead in a state in which a magnetic field is applied to the mold; and binding the inner wall cell and the antibody-coated magnetic bead, and according to the production method, a magnetic cell in a form in which the magnetic bead is attached to a cell outer surface may be produced.

In an exemplary embodiment of the present disclosure, preparing of the inner wall cell of the pipe with fluid flow may include dedifferentiating cells collected from a user into an induced pluripotent stem cell and differentiating the induced pluripotent stem cell into the inner wall cell of the pipe with fluid flow. Specifically, dedifferentiation of an autologous cell collected from a user using a retrovirus is induced to form an induced pluripotent stem cell (hiPSC). The induced pluripotent stem cell may be differentiated into various organ cells such as heart cells, muscle cells vascular endothelial cells, or organ endothelial cells related to diseases in the pipe with fluid flow, and the inner wall cell of the pipe with fluid flow may be prepared by the differentiation.

In an exemplary embodiment of the present disclosure, the autologous cell is collected from user's urine and may be a renal epithelial cell. When a urine sample is used, non-invasive and convenient collection is possible and many cells may be collected, deviating from an invasive method performed in the conventional somatic cell collection.

The dedifferentiation into an induced pluripotent stem cell and the differentiation of an induced pluripotent stem cell using retrovirus may be easily carried out by a person skilled in the art according to a common technology known in the art, and thus, detailed description thereof will be omitted.

Thereafter, the magnetic bead is coated with an inner wall cell-specific antibody. Here, the magnetic bead refers to a particle or bead reactive to the magnetic field and may include a paramagnetic material, a superparamagnetic material, or a ferromagnetic material, but may include a ferromagnetic material for forming a permanent magnet.

In an exemplary embodiment, the magnetic bead may be doped with a low-molecular weight material such as a citric acid or oleic acid for improving a dispersion force, a difunctional carboxylic acid such as mercaptosuccinic acid or hydrocarboxylic acid and a derivative thereof, a synthetic polymer material such as polyethylene glycol, polyvinyl pyrrolidone, polyethyleneimine, polymethacrylate, or polyvinyl alcohol, or a natural polymer material such as polysaccharide. In some embodiments, the magnetic bead may be doped with a biocompatible natural polymer material for in-vivo use, but is not necessarily limited thereto as long as it has a material having biocompatibility.

As an exemplary embodiment of the present disclosure, the magnetic bead may be a magnetic bead having a coating layer formed on the surface, the coating layer being any one or more selected from the group consisting of dextran, carboxymethyl dextran, cellulose, chitin, alginate, starch, and agarose. In addition, the magnetic bead may have a structure to which stepharin, protein A, protein G, protein A/G, or its incorporated functional group is bound for binding to an antibody.

Protein G is a cell wall protein separated from Group C or G streptococcus bacteria (Streptococci.) and an immunoglobulin binding protein having a high binding capacity on an Fc portion of most immunoglobulins, and protein A is a cell wall protein separated from Staphylococcus aureus and may be bound to immunoglobulins expressed in most mammals. The protein G or protein A may be used to impart orientation to the magnetic bead at the time of antibody coating.

After the magnetic bead is coated with the antibody according to the method described above, fixing of the cell to a mold so that a portion of cell is exposed is performed. The mold may be a common mold made of silicon, but is not particularly limited thereto.

Specifically, for example, the mold is filled with the cells so that the cells are half submerged and the magnetic beads coated with the antibody are sprinkled on a mold surface in a state of applying a weak magnetic field to the mold to treat a cell surface with the magnetic bead. Here, the magnetic field may be only intensity for arranging the magnetic bead, and specifically 5 to 10 Gauss. The magnetic bead forms antigen-antibody binding with the antigen on the cell surface by the coated antibody, thereby obtaining the magnetic cell having the magnetic material attached to the cell surface. The magnetic material maintains a non-magnetic paramagnetic state at first for preventing cells from sticking together, and after being injected into the body, is exposed to a magnetic field more than a certain strength to be deformed into a permanent magnetic, ferromagnetic state. The magnetic field only has a degree at which the magnetic material is magnetized, and specifically, 70 Gauss or more.

The magnetic bead may have an average particle diameter of 50 to 1000 nm. The average particle diameter may be 100 to 700 nm, or 300 to 600 nm.

In another general aspect, a system for controlling a shape of a pipe with fluid flow includes the magnetic cell described above and a magnetic field application device.

The magnetic cell is injected in vivo to be released to the inner wall of the pipe with fluid flow to represent a repulsive force or attractive force, thereby causing the expansion or contraction of the pipe with fluid flow to control the shape of the pipe with fluid flow. Here, the magnetic field application device allows the magnetic cell to move to a more correct lesion site and also may provide a hyperthermia effect by an alternating magnetic field. The thermal effect by magnetic field application is to minimize risk of burns or destruction of normal tissues during hyperthermia since the treatment is performed using a magnetic field harmless to the human body.

In addition, in an exemplary embodiment of the present disclosure, the system for controlling a shape of a pipe with fluid flow may further include a near-infrared irradiation device. The near-infrared irradiation device induces local photothermal treatment effect in stenosis in a pipe with fluid flow, thereby providing an increased treatment effect in addition to the hyperthermia effect by the alternating magnetic field application, and may be used in more easily controlling the shape of the pipe with fluid flow.

In an exemplary embodiment of the present disclosure, the system for controlling a shape of a pipe with fluid flow may further include an optical coherence tomography (OCT) device to observe image information of a magnetic cell bound to a pipe with fluid flow in real time after applying a magnetic field by the magnetic field application device, thereby providing feedback information for controlling the shape of the present disclosure.

Specifically, since ferromagnetism is imparted to the magnetic cell injected in vivo by the magnetic field application device to attach the magnetic cell to the inner wall of the pipe with fluid flow, information on the tissue to which the magnetic cell is attached and a subsequent shape change of the pipe with fluid flow may be confirmed from a three-dimensional image by an optical coherence tomography device. Here, a light wavelength may be in a range of 900 to 1300 nm.

In some embodiments, the optical coherence tomography device is connected to the near-infrared irradiation device, and the image information may be analyzed from the light which is reflected back from the tissue after the tissue is irradiated with near infrared. Accordingly, the success or failure of the control of the shape of the pipe with fluid flow using the magnetic cell according to the present disclosure injected into the pipe with fluid flow may be confirmed in real time by a non-invasive method. Therefore, feedback for controlling the shape of the pipe with fluid flow may be easily obtained.

Also, the present disclosure provides an artificial intelligence planning system for controlling a shape of a pipe with fluid flow 100 using the magnetic cell described above. Specifically, the artificial intelligence planning system for controlling a shape of a pipe with fluid flow using a magnetic cell includes: a database unit 110 which includes personal information, medical image information, and shape information of a pipe with fluid flow of existing patients with a disease in a pipe with fluid flow; an interface unit 120 which receives input of biometric information, medical image information, and shape factors of the pipe with fluid flow of patients with a disease in the pipe with fluid flow and outputs predetermined results; a simulation unit 130 which analyzes the information received from the database unit 110 and the interface unit 120 by an artificial intelligence technique to predict a patient's situation after injection of the magnetic cell according to the technical idea described above; and a control planning unit 140 which determines an injection method and an injection site of the magnetic cell depending on the results of the simulation unit and calculates an injection amount.

The personal information of patients may be information such as age, gender, and whether the patient has any underlying disease, and the medical image information may be image data obtained by photographing the body by a medical imaging device, specifically, such as computed tomography (CT), magnetic resonance imaging (MRI), positron emission tomography (PET), and computed radiography (CR). The shape factor of the pipe with fluid flow may be information on the structure or shape of a pipe with fluid flow confirmed from the medical image information or information later required for determining an injection method, an injection site, and an injection amount for controlling the shape of the pipe with fluid flow. In addition, patient's biometric information may be patient's simple physiological data such as blood pressure and heart rate.

Specifically, the simulation unit 130 may perform simulation of the pipe with fluid flow by a basic computational fluid interpretation equation of the Lattice Boltzmann method (LBM). In order to approach the real living environment as close as possible, conditions such as heart rate-based flow rate, blood flow resistance values, fluid viscosity, and elasticity of the pipe with fluid flow which are fitted to the characteristics of each pipe with fluid flow are included in the simulation, and an equation for a correlation of a change in the shape of the pipe with fluid flow depending on a magnetic force is included. Thus, the results from running the simulation assuming the situation after injecting the magnetic cells are reflected in the control planning unit 140 to allow planning optimized for injecting the magnetic cells according to the present disclosure.

The present disclosure has an advantage of predicting control of the shape of the patient's pipe with fluid flow according to artificial intelligence deep learning algorithm based on the clinical data from existing patients.

The disease in the pipe with fluid flow may be any one or more selected from the group consisting of carotid artery stenosis, cerebral aneurysm, dilated cardiomyopathy, abdominal aneurysm, iliac aneurysm, varicose veins, urethral stenosis, prostatic hyperplasia, coronary artery stenosis, asthma, obstructive sleep apnea, and chronic obstructive pulmonary disease.

Depending on the results from the simulation unit, in the case of a mild or acute disease, the magnetic cells may be injected by direct injection of the magnetic cells in a liquid injection form into an affected area for rapid spread of the magnetic cells. In the case of a severe or chronic disease, the magnetic cells may be injected by inserting the magnetic cells into a soft foam and transplanting the foam into the pipe with fluid flow.

Specifically, the method of direct injection into an affected area may be mixing the magnetic cells with a suspension and directly injecting the liquid to the pipe with fluid flow such as a blood vessel or the inside of a respiratory tract. The suspension may be biodegradable PDMS, hyaluronic acid, collagen, chitin, chitosan, heparin, or a combination thereof. It is possible to control a diffusion rate of an injection including the magnetic cells and a retention time of the injected magnetic cells by adjusting the viscosity of the suspension. In addition, at this time, self-control is possible by adjusting an amount of the magnetic cells mixed in the suspension.

When injecting the suspension, a magnetic field may be applied to regulate magnetic force direction control of the injected cells. Specifically, for example, cells in a paramagnetic state having no magnetic force direction determined are injected, and then a certain magnetic field predicted from data analysis is applied to convert the cells into cells in a ferromagnetic state having a magnetic force in a desired direction. The direction and strength of the magnetic field are adjusted for every site to which each cell is injected, thereby precisely controlling the attractive force or repulsive force between the magnetic cells.

In addition, the present disclosure provides a ferromagnetic cell cluster including a plurality of paramagnetic magnetic cells, wherein the magnetic cells form a circular ferromagnetic cell cluster and the ferromagnetic cell cluster is attached to an elastic pipe inner wall to expand or contract a diameter of the elastic pipe by magnetic field application.

In the present disclosure, the “paramagnetic cell” is a magnetic cell including a magnetic material and may refer to a magnetic cell having nonmagnetic properties before applying a magnetic field.

In the present disclosure, an “elastic pipe” refers to a pipe in which in-vivo blood, air, food, waste, and the like flow and elasticity is provided so that a pipe diameter is easily changed. In some embodiments, it may be a blood vessel, a respiratory tract, an esophagus, or a urethra of animals other than humans, but as described above, it is not particularly limited as long as it is a biological pipe in which liquid and gas may flow.

The ferromagnetic cell cluster represents magnetism without external magnetic field application except first magnetization like a permanent magnet, and may adjust an in vivo residence time to be short or long depending on the intensity of the magnetic field to be applied to the paramagnetic cell or the injection method as required. Specifically, for example, in the case of injection in a liquid injection form, the residence time is relatively short, and in the case of in-vivo insertion of a foam after a liquid phase is absorbed in a soft foam, long-term retention in the living body is possible.

As a method of importing magnetism to the inner wall cell of the pipe with fluid flow, according to the present disclosure, an immunolabeling or direct introduction method using an antigen-antibody reaction is possible. The immunolabeling for imparting magnetism will be described in more detail by the following examples.

Hereinafter, the magnetic cell for controlling a shape of a pipe with fluid flow according to the present disclosure will be described in more detail by the following examples. However, the following exemplary embodiments are only a reference for describing the present disclosure in detail, and the present disclosure is not limited thereto, and may be implemented in various forms.

### Preparation Example 1

Production of Inner Wall Cell of Pipe with Fluid Flow by Dedifferentiation of Autologous Cell

10 cc of user's first urine was collected and centrifuged at 1500 rpm at 4° C. for 5 minutes, a supernatant was removed, and then renal epithelial cells were collected from the precipitate.

A DMEM medium supplemented with 10% purified bovine serum (FBS), 100 U/ml of penicillin, and 100 ug/ml of streptomycin was added to a plate, and separated renal epithelial cells were inoculated at 1×10⁴ to 1×10⁵ cells/ml. Subsequently, a retrovirus was added at 1 MOI, and incubation was performed under conditions of 37° C. and 5% CO₂. The process was repeated three times once every three days. On the 12th day, it was replaced with a DMEM-12 medium supplemented with a human embryonic stem cell culture medium containing bFGF, 100 U/ml of penicillin, 100 ug/ml of streptomycin, and 4 ng/ml of bFGF and incubation was performed to obtain an iPS cell colony after 5 days. A 1×10⁷ cells iPS cell colony was incubated and differentiated to produce thoracic aortic inner wall cells.

### Example 1

Production of Magnetic Cell by Immunolabeling Based on Immune Reaction

10 mg of 500 nm-sized beads having magnetism coated with dextran on the surface was placed into a 2 ml tube, and a phosphate buffer solution at pH 5.5 was added and mixed. Thereafter, the mixture was placed in a magnetic separator to collect magnetic beads and remove a suspension, and the precipitated magnetic beads were washed. 200 μg/ml of desalted and purified anti-mouse IgG was added in 1 ml each to the remaining magnetic beads and mixed, and stirred at 4° C. for 15 hours. Thereafter, the magnetic beads to which the anti-IgG was bound were separated with a magnetic separator, were washed three times with a 0.05 M Tris-HCl solution, and were dispersed in 1 ml of a PBS buffer solution containing 0.1% BSA to produce antibody-coated magnetic beads. Thoracic aortic inner wall cells produced according to Preparation Example 1 were fixed to a silicon mold so that the cells were half submerged, about 40 μWb of magnetic field was applied, and the magnetic beads coated with the antibody were sprinkled on the surface of the mold to bind the antibody of the inner wall cells of the pipe with fluid flow and the antibody coated on the magnetic beads, thereby producing magnetic cells having a magnetic material attached on the surface.

### Experimental Example 1

Effect of Controlling Shape of Pipe with Fluid Flow Depending on Running of System for Controlling Shape of Pipe with Fluid Flow

A thoracic aortic model having blood vessel stenosis (T-S-N 005, Geneva, Elastrat) was prepared. The blood vessel model was designed to have a basic diameter of 2 mm and a stenosis site diameter of 1 mm, the magnetic cells produced according to Example 1 were mixed in a PDMS solution at a concentration of 1×10⁷ cells/ml, and 10 ml of the solution was injected into upper and lower portions of the stenosed inner wall of the aortic model. A heart rate-based flow rate, a blood flow resistance value, and a flow rate waveform were controlled by a control unit (CardioFlow 5000 MR, Shelley Medical Imaging Technologies), a magnetic field application device was used to apply 80 Gauss magnetic field to convert the injected magnetic cells into a ferromagnetic state, and a repulsive force between the injected magnetic cells was caused. Accordingly, the results of the expanded diameter of the stenosed blood vessel to 1.6 mm were confirmed.

While the exemplary embodiment of the present disclosure has been described above, the present disclosure is not limited thereto, and various modification may be carried out, the description of the disclosure, and the attached drawings, which also belongs to the scope of the present disclosure, of course.

### DETAILED DESCRIPTION OF MAIN ELEMENTS

100: Artificial intelligence planning system for controlling a shape of a pipe with fluid flow using magnetic cells
110: Database unit
120: Interface unit
130: Simulation unit
140: Control planning unit

1. A magnetic cell for controlling a shape of a pipe with fluid flow, the magnetic cell comprising a magnetic material in the cell.

2. The magnetic cell for controlling a shape of a pipe with fluid flow of claim 1, wherein the cell is an inner wall cell of the pipe with fluid flow.

3. The magnetic cell for controlling a shape of a pipe with fluid flow of claim 1, wherein the magnetic material is a permanent magnet.

4. The magnetic cell for controlling a shape of a pipe with fluid flow of claim 1, wherein the magnetic material is bound to a cell surface.

5. The magnetic cell for controlling a shape of a pipe with fluid flow of claim 1, wherein the magnetic material is placed inside the cell.

6. The magnetic cell for controlling a shape of a pipe with fluid flow of claim 1, wherein controlling a shape is to expand or contract the pipe with fluid flow using a repulsive force or attractive force between the magnetic cells.

7. The magnetic cell for controlling a shape of a pipe with fluid flow of claim 1,
wherein the pipe with fluid flow is a blood vessel, a urethra, a respiratory tract, or an esophagus.

8. A method of producing a magnetic cell for controlling a shape of a pipe with fluid flow, the method comprising:
preparing an inner wall cell of a pipe with fluid flow;
coating a magnetic bead with an inner wall cell-specific antibody;
fixing the cell to a mold so that a portion of the inner wall cell is exposed;
treating a surface of the inner wall cell with the antibody-coated magnetic bead in a state in which a magnetic field is applied to the mold; and
binding the inner wall cell and the antibody-coated magnetic bead.

9. The method of producing a magnetic cell for controlling a shape of a pipe with fluid flow of claim 8,
wherein the preparing of an inner wall cell of a pipe with fluid flow includes: dedifferentiating cells collected from a user into an induced pluripotent stem cell and differentiating the induced pluripotent stem cell into the inner wall cell of the pipe with fluid flow.

10. The method of producing a magnetic cell for controlling a shape of a pipe with fluid flow of claim 8,
wherein the magnetic bead has an average particle diameter of 50 to 1000 nm.

11. A system for controlling a shape of a pipe with fluid flow comprising the magnetic cell of claim 1 and a magnetic field application device.

12. The system for controlling a shape of a pipe with fluid flow of claim 11, further comprising:
an optical coherence tomography (OCT) device to observe image information of the magnetic cell bound to the pipe with fluid flow in real time after applying a magnetic field by the magnetic field application device, thereby providing feedback information for controlling the shape of the pipe with fluid flow.

13. The system for controlling a shape of a pipe with fluid flow of claim 11, further comprising:
a near-infrared irradiation device.

14. An artificial intelligence planning system for controlling a shape of a pipe with fluid flow using a magnetic cell comprising:
a database unit which includes personal information, medical image information, and shape information of a pipe with fluid flow of existing patients with a disease in a pipe with fluid flow;
an interface unit which receives input of biometric information, medical image information, and shape factors of the pipe with fluid flow of patients with a disease in a pipe with fluid flow and outputs predetermined results;
a simulation unit which analyzes the information received from the database unit and the interface unit by an artificial intelligence technique to predict a patient's situation after injection of the magnetic cell of claim 1; and
a control planning unit which determines an injection method and an injection site of the magnetic cell depending on the results of the simulation unit and calculates an injection amount.

15. The artificial intelligence planning system for controlling a shape of a pipe with fluid flow using a magnetic cell of claim 14, wherein
the injection method of the magnetic cell is direct injection into an affected area.

16. The artificial intelligence planning system for controlling a shape of a pipe with fluid flow using a magnetic cell of claim 14,
wherein the injection method of the magnetic cell is insertion of the magnetic cell into a soft foam and transplantation of the foam into the pipe with fluid flow.

17. The artificial intelligence planning system for controlling a shape of a pipe with fluid flow using a magnetic cell of claim 14,
wherein the disease in the pipe with fluid flow is any one or more selected from the group consisting of carotid artery stenosis, cerebral aneurysm, dilated cardiomyopathy, abdominal aneurysm, iliac aneurysm, varicose veins, urethral stenosis, prostatic hyperplasia, coronary artery stenosis, asthma, obstructive sleep apnea, and chronic obstructive pulmonary disease.

18. A ferromagnetic cell cluster comprising the plurality of paramagnetic cells of claim 1, wherein the magnetic cells form a circular ferromagnetic cell cluster and the ferromagnetic cell cluster is attached to an elastic pipe inner wall to expand or contract a diameter of the elastic pipe by magnetic field application.
